(19) United States
(12) Patent Application Publication
(10) Pub. No.: US 2022/0133996 A1
(43) Pub. Date: May 5, 2022

(54) CAMMED ROLLER BEARING TUBING CLAMP

(71) Applicant: BAYER HEALTHCARE LLC, WHIPPANY, NJ (US)

(72) Inventor: GERALD DELBRUGGE, CLEARFIELD, PA (US)

(21) Appl. No.: 17/602,062

(22) PCT Filed: May 1, 2020
(86) PCT No.: PCT/US2020/030957

§ 371 Date: Oct. 7, 2021

Related U.S. Application Data
(60) Provisional application No. 62/842,881, filed on May 3, 2019.

(51) Int. Cl. A61M 5/168; A61M 39/28

(52) U.S. Cl. CPC A61M 5/16813; A61M 5/1452; A61M 39/285

(57) ABSTRACT

A clamp for regulating fluid flow in a flexible tube of a fluid injector system including an anvil and a cam assembly is described. The anvil includes a receiving surface for receiving the flexible tube. The cam assembly includes either a solid cam or a rollable outer race and an inner race having a central axis and a rotation axis spaced apart from the central axis. The inner race is rotatable relative to the outer race. Rotation of the inner race about the rotation axis rolls the outer race in a direction relative to the receiving surface of the anvil to reversibly compress the flexible tube between the outer race and the anvil.

### CROSS-REFERENCE TO RELATED APPLICATIONS

The present application claims the benefit of U.S. Provisional Patent Application No. 62/842,881, filed on May 3, 2019, the disclosure of which is hereby incorporated by reference in its entirety.

### BACKGROUND OF THE DISCLOSURE

### Field of the Disclosure

The present disclosure is related to the field of tubing clamps for a medical injector system. In particular, the tubing clamps of the present disclosure are capable of effectively stopping fluid flow through a fluid path set between a medical injector and a patient without placing undue stress on injector components and fluid paths.

### Description of Related Art

In many medical diagnostic and therapeutic procedures, a patient is injected with one or more fluids. In recent years, a number of injector-actuated syringes and powered injectors for pressurized injection of fluids have been developed for use in procedures such as angiography (CV), computed tomography (CT), molecular imaging (such as PET imaging), and magnetic resonance imaging (MRI). In these procedures, a medical fluid, such as a contrast agent, may be used to highlight certain vasculature systems, internal organs, or portions of the body during an imaging process. The medical fluid may be delivered to the patient by the powered injector by one or more pump, syringe, or combination thereof.

When preparing to inject a medical fluid into a patient, it is important that the injection reservoir is fully filled with the medical fluid and air removed to avoid inadvertent injection of air into the patient. In certain procedures such as angiography, even small quantities of air may present a concern if injected into the vasculature during the injection procedure. The inclusion of air detectors, either at the syringe or on the fluid path may help notify the user that air is present and there is a possibility of the air being injected with the contrast. When air is detected, stopping the injection procedure prior to the air reaching the patient's vasculature is desired. However, due to system pressurization resulting in compliance, i.e., swelling or deflection of system components particularly at pressures used for injection of medical fluids during certain procedures, simply stopping the injection by stopping the motor of the powered injector may not immediately stop the flow of fluid through the fluid path set and into the patient. Further, during simultaneous injection of two or more fluids (“dual flow injection”), differences in fluid viscosity and pressure of the fluids may result in backflow of one fluid into the fluid path and reservoir of the other fluid, resulting in dilution of the second fluid and volume inaccuracies during fluid injection and/or decreased image properties. Therefore, devices and methods that rapidly and effectively stop the fluid flow and prevent backflow during injection procedures.

### SUMMARY OF THE DISCLOSURE

In view of the foregoing, there exists a need for devices and methods for rapidly stopping the flow of fluid in tubing, such as medical injector tubing during a powered fluid injection procedure. Accordingly, some aspects or examples of the present disclosure are directed to a clamp for regulating fluid flow in a flexible tube of a fluid injector system. The clamp includes an anvil including a receiving surface for receiving at least a portion of the flexible tube, and a cam assembly. The cam assembly includes a rollable outer race, and an inner race having a central axis and a rotation axis spaced apart from the central axis. The inner race is rotatable relative to the outer race about the central axis. Rotation of the inner race about the rotation axis rolls the outer race in a direction relative to the receiving surface of the anvil to reversibly compress the flexible tube between the outer race and the anvil.

In some aspects or examples, the inner race of the cam assembly is rotatable to a first position in which the flexible tube is substantially uncompressed between the outer race and the anvil such that fluid can flow through a lumen of the flexible tube; and a second position in which the lumen of the flexible tube is fully compressed between the outer race and the anvil to prohibit fluid flow through the flexible tube, such that fluid communication across a compressed region of the flexible tube is blocked.

In some aspects or examples, the inner race of the cam assembly is rotatable to at least partially compress the flexible tube between the outer race and the anvil, thereby controlling a flow rate of fluid through an at least partially compressed region of the flexible tube.

In some aspects or examples, a compression force on the flexible tube between the outer race and the anvil is from about 0.1% to about 100% of a rolling compression force and from about 99.9% to about 0% of a sliding compression force.

In some aspects or examples, the cam assembly further includes a plurality of rolling elements between the inner race and the outer race.

In some aspects or examples, the central axis and the rotation axis of the inner race extend perpendicular to a longitudinal axis of the flexible tube.

In some aspects or examples, the central axis and the rotation axis of the inner race extend parallel to a longitudinal axis of the flexible tube.

In some aspects or examples, the receiving surface of the anvil defines a groove for receiving at least a portion of the flexible tube.

In some aspects or examples, the outer race includes at least one annular protrusion cooperative with the groove of the receiving surface.

In some aspects or examples, the clamp further includes a motor having a shaft for rotating the inner race about the rotation axis.

In some aspects or examples, wherein at least a portion of the outer race has a textured surface to prevent slippage between the outer race and flexible tube.

In some aspects or examples, the textured surface of the outer race is directional to prevent rotation of the outer race away from the flexible tube.

In some aspects or examples, the anvil defines a detent, wherein rotation of the inner race about the rotation axis compresses the flexible tube between the outer race and the detent of the anvil.

In some aspects or examples, at least a portion of a section of the receiving surface of the anvil is substantially planar.

In some aspects or examples, a contact point of the outer race at which the outer race engages the flexible tube remains in constant contact with the flexible tube as the flexible tube is reversibly compressed.

Other aspects or examples of the present disclosure are directed to a fluid injector system including at least one injector for pressurizing and delivering at least one fluid from at least one fluid reservoir, a fluid path set including at least one flexible tube in fluid communication with the at least one fluid reservoir and, a controller; and at least one clamp for regulating fluid flow through the at least one flexible tube of the fluid path set. The at least one clamp includes an anvil including a receiving surface for receiving at least a portion of the at least one flexible tube, and a cam assembly. The cam assembly includes a rollable outer race, and an inner race having a central axis and a rotation axis spaced apart from the central axis. The inner race is rotatable relative to the outer race about the central axis. Rotation of the inner race about the rotation axis rolls the outer race in a direction relative to the receiving surface of the anvil to reversibly compress the flexible tube between the outer race and the anvil. The controller is programmed or configured to control rotation of the inner race of the cam assembly.

In some aspects or examples, the controller is programmed or configured to rotate the inner race of the cam assembly to a first position in which the flexible tube is substantially uncompressed between the outer race and the anvil such that fluid can flow through a lumen of the flexible tube; and a second position in which the lumen of the flexible tube is fully compressed between the outer race and the anvil to prohibit fluid flow through the flexible tube, such that fluid communication across a compressed region of the flexible tube is blocked.

In some aspects or examples, the controller is programmed or configured to rotate the inner race of the cam assembly to at least partially compress the flexible tube between the outer race and the anvil, thereby controlling a flow rate of the at least one fluid through an at least partially compressed region of the flexible tube.

In some aspects or examples, the fluid injector system further includes at least one air detector for detecting a presence of air in the fluid path set. The at least one clamp is located downstream of the at least one air detector. The controller is programmed or configured to rotate the inner race of the cam assembly to the second position in response to detecting the presence of air in the fluid path set by the at least one air detector to prevent flow of the air past the at least one clamp.

In some aspects or examples, the controller is further programmed or configured to rotate the inner race of the cam assembly to the second position to prevent backflow of at least one first fluid into one or more of at least one second fluid reservoirs or a second fluid tube.

Other aspects or examples of the present disclosure are directed to a clamp for regulating fluid flow in a flexible tube of a fluid injector system. The clamp includes an anvil including a receiving surface for receiving the flexible tube, and a cam assembly. The cam assembly includes a rollable solid cam having central axis and a rotation axis spaced apart from the central axis. Rotation of the rollable solid cam about the rotation axis rolls the rollable solid cam in a direction relative to the receiving surface of the anvil to reversibly compress the flexible tube between the rollable solid cam and the anvil.

In some aspects or examples, the rollable solid cam is rotatable to a first position in which the flexible tube is uncompressed between the rollable solid cam and the anvil such that fluid can flow through a lumen of the flexible tube; and a second position in which the lumen of the flexible tube is fully compressed between the rollable solid cam and the anvil to prohibit fluid flow through the flexible tube, such that fluid communication across a compressed region of the flexible tube is blocked.

In some aspects or examples, the rollable solid cam is rotatable to at least partially compress the flexible tube between the rollable solid cam and the anvil, thereby controlling a flow rate of fluid through an at least partially compressed region of the flexible tube.

In some aspects or examples, a compression force on the flexible tube between the rollable solid cam and the anvil is from about 0.1% to about 100% of a rolling compression force and from about 99.9% to about 0% of a sliding compression force.

Further aspects or examples of the present disclosure are described in the following numbered clauses:

Clause 1. A clamp for regulating fluid flow in a flexible tube of a fluid injector system, the clamp comprising: an anvil comprising a receiving surface for receiving at least a portion of the flexible tube; and a cam assembly comprising: a rollable outer race; and an inner race having a central axis and a rotation axis spaced apart from the central axis, wherein the inner race is rotatable relative to the outer race about the central axis, wherein rotation of the inner race about the rotation axis rolls the outer race in a direction relative to the receiving surface of the anvil to reversibly compress the flexible tube between the outer race and the anvil.

Clause 2. The clamp of clause 1, wherein the inner race of the cam assembly is rotatable to: a first position in which the flexible tube is substantially uncompressed between the outer race and the anvil such that fluid can flow through a lumen of the flexible tube; and a second position in which the lumen of the flexible tube is fully compressed between the outer race and the anvil to prohibit fluid flow through the flexible tube, such that fluid communication across a compressed region of the flexible tube is blocked.

Clause 3. The clamp of clause 1 or 2, wherein the inner race of the cam assembly is rotatable to at least partially compress the flexible tube between the outer race and the anvil, thereby controlling a flow rate of fluid through an at least partially compressed region of the flexible tube.

Clause 4. The clamp of any of clauses 1 to 3, wherein a compression force on the flexible tube between the outer race and the anvil is from about 0.1% to about 100% of a rolling compression force and from about 99.9% to about 0% of a sliding compression force.

Clause 5. The clamp of any of clauses 1 to 4, wherein the cam assembly further comprises a plurality of rolling elements between the inner race and the outer race.

Clause 6. The clamp of any of clauses 1 to 5, wherein the central axis and the rotation axis of the inner race extend perpendicular to a longitudinal axis of the flexible tube.

Clause 7. The clamp of any of clauses 1 to 6, wherein the central axis and the rotation axis of the inner race extend parallel to a longitudinal axis of the flexible tube.

Clause 8. The clamp of any of clauses 1 to 7, wherein the receiving surface of the anvil defines a groove for receiving at least a portion of the flexible tube.

Clause 9. The clamp of any of clauses 1 to 8, wherein the outer race comprises at least one annular protrusion cooperative with the groove of the receiving surface.

Clause 10. The clamp of any of clauses 1 to 9, further comprising a motor having a shaft for rotating the inner race about the rotation axis.

Clause 11. The clamp of any of clauses 1 to 10, wherein at least a portion of the outer race has a textured surface to prevent slippage between the outer race and flexible tube.

Clause 12. The clamp of any of clauses 1 to 11, wherein the textured surface of the outer race is directional to prevent rotation of the outer race away from the flexible tube.

Clause 13. The clamp of any of clauses 1 to 12, wherein the anvil defines a detent, and wherein rotation of the inner race about the rotation axis compresses the flexible tube between the outer race and the detent of the anvil.

Clause 14. The clamp of any of clauses 1 to 13, wherein at least a portion of a section of the receiving surface of the anvil is substantially planar.

Clause 15. The clamp any of clauses 1 to 14, wherein a contact point of the outer race at which the outer race engages the flexible tube remains in constant contact with the flexible tube as the flexible tube is reversibly compressed.

Clause 16. A fluid injector system comprising: at least one injector for pressurizing and delivering at least one fluid from at least one fluid reservoir; a fluid path set comprising at least one flexible tube, wherein the fluid path set is in fluid communication with the at least one fluid reservoir; a controller; and at least one clamp for regulating fluid flow through the at least one flexible tube of the fluid path set, the at least one clamp comprising: an anvil comprising a receiving surface for receiving at least a portion of the at least one flexible tube; and a cam assembly comprising: a rollable outer race; and an inner race having a central axis and a rotation axis spaced apart from the central axis, wherein the inner race is rotatable relative to the outer race about the central axis, wherein rotation of the inner race about the rotation axis rolls the outer race in a direction relative to the receiving surface of the anvil to reversibly compress the flexible tube between the outer race and the anvil, and wherein the controller is programmed or configured to control rotation of the inner race of the cam assembly.

Clause 17. The fluid injector system of clause 16, wherein the controller is programmed or configured to rotate the inner race of the cam assembly to: a first position in which the flexible tube is substantially uncompressed between the outer race and the anvil such that fluid can flow through a lumen of the flexible tube; and a second position in which the lumen of the flexible tube is fully compressed between the outer race and the anvil to prohibit fluid flow through the flexible tube, such that fluid communication across a compressed region of the flexible tube is blocked.

Clause 18. The fluid injector system of clause 16 or 17, wherein the controller is programmed or configured to rotate the inner race of the cam assembly to at least partially compress the flexible tube between the outer race and the anvil, thereby controlling a flow rate of the at least one fluid through an at least partially compressed region of the flexible tube.

Clause 19. The fluid injector system of any of clauses 16 to 18, further comprising: at least one air detector for detecting a presence of air in the fluid path set, wherein the at least one clamp is located downstream of the at least one air detector, and wherein the controller is programmed or configured to rotate the inner race of the cam assembly to the second position in response to detecting the presence of air in the fluid path set by the at least one air detector to prevent flow of the air past the at least one clamp.

Clause 20. The fluid injector system any of clauses 16 to 19, wherein the controller is further programmed or configured to rotate the inner race of the cam assembly to the second position to prevent backflow of at least one first fluid into one or more of at least one second fluid reservoir or a second fluid tube.

Clause 21. A clamp for regulating fluid flow in a flexible tube of a fluid injector system, the clamp comprising: an anvil comprising a receiving surface for receiving the flexible tube; and a cam assembly comprising: a rollable solid cam having central axis and a rotation axis spaced apart from the central axis, wherein rotation of the rollable solid cam about the rotation axis rolls the rollable solid cam in a direction relative to the receiving surface of the anvil to reversibly compress the flexible tube between the rollable solid cam and the anvil.

Clause 22. The clamp of clause 21, wherein the rollable solid cam is rotatable to: a first position in which the flexible tube is substantially uncompressed between the rollable solid cam and the anvil such that fluid can flow through a lumen of the flexible tube; and a second position in which the lumen of the flexible tube is fully compressed between the rollable solid cam and the anvil to prohibit fluid flow through the flexible tube, such that fluid communication across a compressed region of the flexible tube is blocked.

Clause 23. The clamp of any of clause 21 or 22, wherein the rollable solid cam is rotatable to at least partially compress the flexible tube between the rollable solid cam and the anvil, thereby controlling a flow rate of fluid through an at least partially compressed region of the flexible tube.

Clause 24. The clamp of any of clauses 21 to 23, wherein a compression force on the flexible tube between the rollable solid cam and the anvil is from about 0.1% to about 100% of a rolling compression force and from about 99.9% to about 0% of a sliding compression force.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a perspective view of a fluid injector system according to an aspect or example of the present disclosure;

FIG. 2 is a schematic diagram of the fluid injector system of FIG. 1;

FIG. 3 is a perspective view of a tubing clamp according to an aspect or example of the present disclosure;

FIG. 4 is a side view of the tubing clamp of FIG. 3 in an open position;

FIG. 5 is a side view of the tubing clamp of FIG. 3 in a closed position;

FIG. 6A is a side view of a tubing clamp in the closed position according to another aspect or example of the present disclosure;

FIG. 6B is a side view of the tubing clamp of FIG. 6A in a partially closed position;

FIG. 7 is a side view of a tubing clamp in an open position according to another aspect or example of the present disclosure;

FIG. 8 is a front view of the tubing clamp of FIG. 7 in a closed position.

FIG. 9 is a side view of a tubing clamp in a closed position according to another aspect or example of the present disclosure;

FIG. 10 is a front view of a tubing clamp in an open position according to another aspect or example of the present disclosure;

FIG. 11 is a front view of the tubing clamp of FIG. 10 in a closed position;

FIG. 12 is a side view of a tubing clamp in an open position according to another aspect or example of the present disclosure;

FIG. 13 is a side view of a tubing clamp in an open position according to another aspect or example of the present disclosure;

FIG. 14 is a side view of a tubing clamp in an open position according to another aspect or example of the present disclosure;

FIG. 15 is a side view of a tubing clamp in an open position according to another aspect or example of the present disclosure; and

FIG. 16 is a side view of a tubing clamp in an open position according to another aspect or example of the present disclosure.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe of a multi-patient disposable set, the term “proximal” refers to a portion of a syringe nearest a piston for delivering fluid from a syringe.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a component of a fluid injector system such as a fluid reservoir, a syringe, or a fluid line, the term “distal” refers to a portion of said component nearest to a patient. When used in relation to a component of a fluid injector system such as a fluid reservoir, a syringe, or a fluid line, the term “proximal” refers to a portion of said component nearest to the injector of the fluid injector system (i.e. the portion of said component farthest from the patient). When used in relation to a component of a fluid injector system such as a fluid reservoir, a syringe, or a fluid line, the term “upstream” refers to a direction away from the patient and towards the injector of the fluid injector system. For example, if a first component is referred to as being “upstream” of a second component, the first component is located nearer to the injector than the second component is to the injector. When used in relation to a component of a fluid injector system such as a fluid reservoir, a syringe, or a fluid line, the term “downstream” refers to a direction towards the patient and away from the injector of the fluid injector system. For example, if a first component is referred to as being “downstream” of a second component, the first component is located nearer to the patient than the second component is to the patient.

As used herein, the terms “capacitance” and “impedance” are used interchangeably to refer to a volumetric expansion of injector components, such as fluid reservoirs, syringes, fluid lines, and/or other components of a fluid injector system as a result of pressurized fluids with such components and/or uptake of mechanical slack by force applied to components. Capacitance and impedance may be due to high injection pressures, which may be on the order of 1,200 psi in some angiographic procedures, and may result in a volume of fluid held within a portion of a component in excess of the desired quantity selected for the injection procedure or the resting volume of the component. Additionally, capacitance of various components can, if not properly accounted for, adversely affect the accuracy of pressure sensors of the fluid injector system because the volumetric expansion of components can cause an artificial drop in measured pressure of those components.

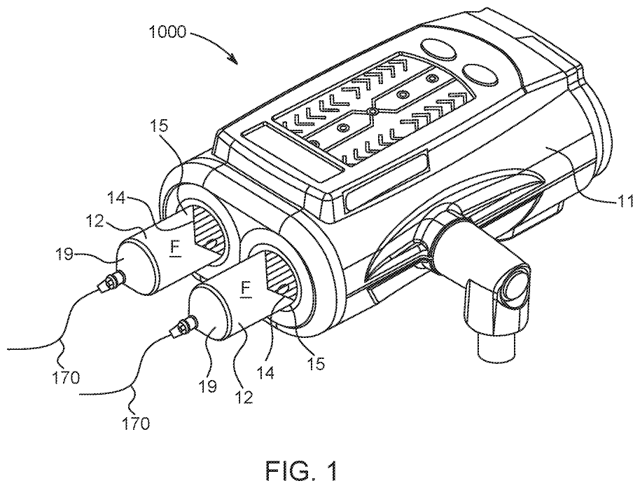

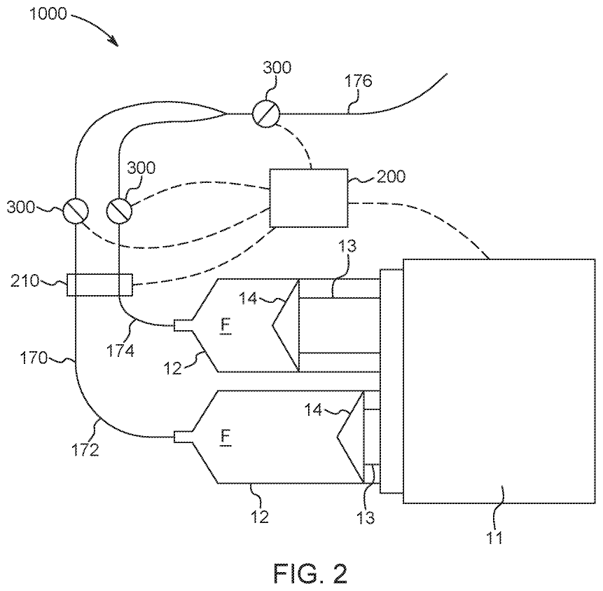

Referring to the drawings in which like reference characters refer to like parts throughout the several views thereof, the present disclosure is generally directed to a clamp for regulating fluid flow in a fluid injector system. Referring first to FIGS. 1 and 2, an example of a fluid injector system 1000 in accordance with the present disclosure includes a housing 11 and at least one fluid reservoir, such as at least one syringe 12. The fluid injector system 1000 further includes a piston 13 associated with each of the syringes 12 that drives a plunger 14 within a barrel of the syringe 12. The at least one syringe 12 is generally adapted to releasably interface with the housing 11 at a syringe port 15. The at least one syringe 12 may be oriented in any manner such as upright, downright, or positioned at any degree angle. The fluid injector system 1000 is generally configured to deliver at least one fluid F to a patient during an injection procedure. The fluid injector system 1000 is configured to releasably receive the at least one syringe 12, which is to be filled with at least one medical fluid F, such as an imaging contrast media, saline solution, or any desired medical fluid. Each syringe 12 may be filled with a different medical fluid F. The fluid injector system 1000 may be a multi-syringe injector, as shown, wherein several syringes 12 may be oriented side-by-side or in another spatial relationship and are separately actuated by respective pistons associated with the injector system 1000.

With continued reference to FIGS. 1 and 2, the fluid injector system 1000 may be used during a medical procedure to inject the at least one medical fluid F into the vasculature of a patient by driving the plungers 14 associated with the at least one syringe 12 with the at least one piston 13. The at least one piston 13 may be reciprocally operable upon the plunger 14. Upon engagement, the at least one piston 13 may move the plunger 14 toward a proximal end of the at least one syringe 12 to draw the medical fluid F into the at least one syringe 12 from a bulk fluid reservoir (not shown), such as a vial, bottle, or intravenous bag. The at least one piston 13 may further move the plunger 14 toward a distal end 19 of the at least one syringe 12 to expel the fluid F from the at least one syringe 12. A fluid path set 170 may include at least one tube or tube set configured to be in fluid communication with each syringe 12 to place each syringe 12 in fluid communication with a flexible administration tube 176 for delivering the fluid F from each syringe 12 to a patient at a vascular access site.

As shown in FIG. 2, the fluid path set 170 may include a first flexible tube 172 fluidly connected to a first of the syringes 12 and a second flexible tube 174 fluidly connected to a second of the syringes 12. The first flexible tube 172 and the second flexible tube 174 of the fluid path set 170 may merge into a distal flexible tube 176 for connection to the patient, or to one or more intervening components, such as an administration flexible tube or catheter, connected to the patient. Each of the first flexible tube 172, the second flexible tube 174, and the distal flexible tube 176 of the fluid path set 170 may be formed of a flexible and reversibly compressible material, such as a polymer. As used hereon, the term “reversibly compressible” means that the cross-sectional shape of the flexible tube of the fluid path set 170, or a portion thereof, can change by applying a force thereto, and upon release of the applied force, the flexible tube of the fluid path set 170 returns to its original shape. For example, a force may be applied to an outside surface of the flexible tube of the fluid path set 170, causing the diametrically opposed points on the inner sidewall of the flexible tube of the fluid path set 170 to be brought together to alter the cross-sectional area of a lumen of flexible tube of fluid path set 170.

With continued reference to FIG. 2, the fluid injector system 1000 may further include a controller 200 for controlling actuation of the at least one piston 13 and other components of the fluid injector system 1000. The fluid injector system 1000 may perform one or more injection procedures according to one or more injection protocols stored in a memory of or accessible by the controller 200. The controller 200 may communicate with at least one air detector assembly 210 configured to detect the presence of air in the fluid path set 170. The controller 200 may be configured to stop actuation of the at least one syringe 12 in response to the air detector assembly 210 detecting air in the tubing of the fluid path set 170 in order to prevent air from being injected into the patient. Stopping actuation of the at least one syringe 12 may include halting distal movement of the piston 13 of the at least one syringe 12 and/or moving at least one valve, such as a rolling cam pinch valve of the present disclosure to a closed position wherein a portion of the downstream tubing is compressed to prevent fluid flow past the at least one valve.

Further details and examples of suitable nonlimiting powered injector systems, including syringes, controllers, air detectors, and fluid path sets are described in U.S. Pat. Nos. 5,383,858; 7,553,294; 7,666,169; 8,945,051; 10,022,493; and 10,507,319, the disclosures of which are hereby incorporated by reference in their entireties.

With continued reference to FIG. 2, the fluid injector system 1000 may further include one or more tubing clamps or valves 300 disposed at various locations along the fluid path set 170. Each of the tubing clamps 300 may be in the form of a shut-off valve and/or a flow rate control valve to regulate flow of the medical fluid F to the patient. In the aspect or example shown in FIG. 2, one of the tubing clamps 300 is provided on each of the first flexible tube 172, the second flexible tube 174, and the distal flexible tube 176 of the fluid path set 17. In some aspects or examples, a tubing clamp 300 may be provided only on the distal flexible tube 176 of the fluid path set 17. In some aspects or examples, one or more of the tubing clamps 300 may be mounted directly to the housing 11 or other component of the fluid injector system 1000.

Each of the tubing clamps 300 may be controllable by the controller 200 to regulate the flow of the fluid F through the fluid path set 170. For example, any or all of the tubing clamps 300 may be closed by the controller 200 in response to the at least one air detector assembly 210 detecting air in the fluid path set 170. Closure of each tubing clamp 300 reversibly compresses the flexible tubing 172, 174, and/or 176 of the fluid path set 170 to halt fluid flow through the fluid path set 170. Closure of the tubing clamps 300 in this manner prevents the medical fluid F from advancing downstream of the clamps 300, thereby preventing air from being injected into the patient due to relief of capacitance within the fluid path set 170 and/or the syringes 12. In contrast, only the halting the movement of the at least one piston 13 may allow the medical fluid F, and any air contained therein, to be injected into the patient as capacitance in the fluid path set 170 and/or the syringes 12 is relieved and the released volume of fluid F flows through the tubing.

The tubing clamps 300 may alternatively or additionally be utilized to perform functions other than halting fluid flow in response to air detection. In some aspects or examples, the tubing clamp 300 provided on the first flexible tube 172 of the fluid path set 170 may be closed by the controller 200 to prevent backflow of pressurized medical fluid F from the second flexible tube 174 and/or second syringe 12 into the first flexible tube 172 or first syringe due to a difference in pressure between the two syringes and associated tubing. Similarly, a tubing clamp 300 on the second flexible tube 174 may be closed to prevent backflow from a higher pressurized fluid in the first flexible tube 172 and/or first syringe 12. In some aspects or examples, any or all of the tubing clamps 300 may be partially closed by the controller 200 to limit or control a flow rate of the medical fluid F in accordance with an injection protocol. For example, partially the closing tubing clamp 300 associated with the first flexible tube 172 may decrease the fluid flow of the first fluid through the first flexible tube 172. The reduction in fluid flow rate may be calculated according to an algorithm with variables associated with percent fluid path closure, fluid pressure, upstream tubing capacitance, fluid viscosity, pressure drop across the clamp, and the like.

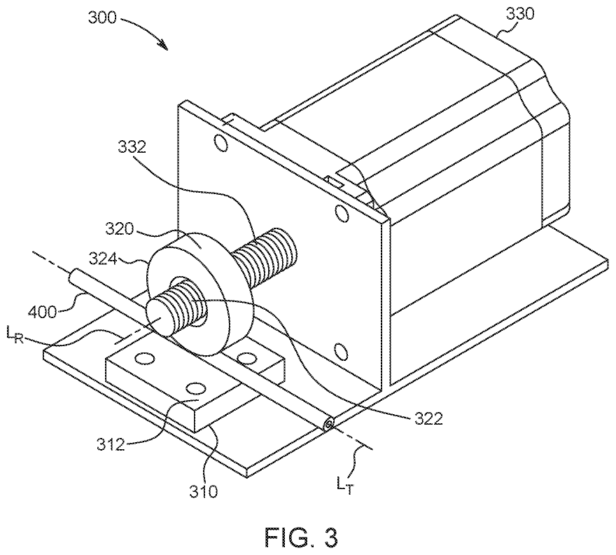

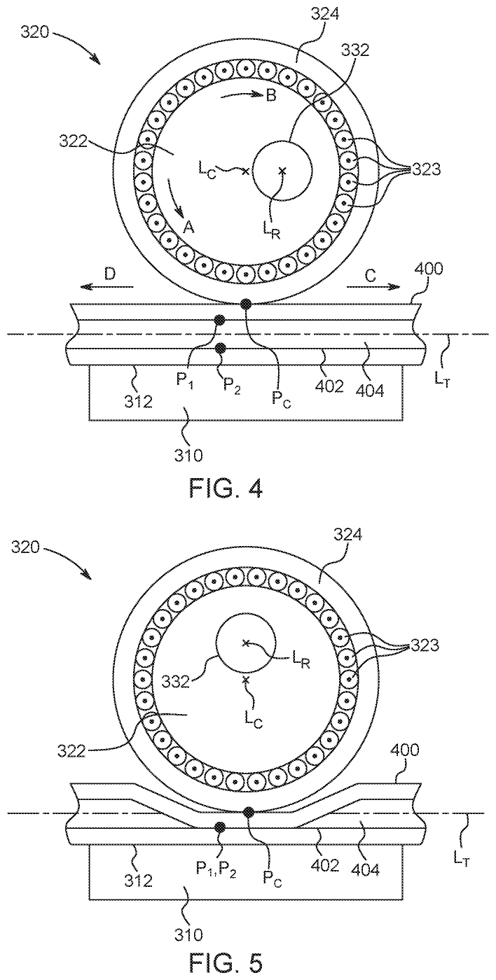

Having generally described the fluid injector system 1000, further detail of the tubing clamp 300 will now be provided with reference to FIGS. 3-16. According to various embodiments herein, the present disclosure provides for a tubing clamp in the form of a cammed rolling tube clamp. Referring first to the aspect or example shown in FIGS. 3-5, the tubing clamp 300 includes an anvil 310 and an eccentrically rotating cam assembly 320. FIG. 4 shows the tubing clamp 300 in an open position, while FIG. 5 shows the tubing clamp 300 in a closed position. The anvil 310 includes a receiving surface 312 for receiving at least a portion of the flexible tubing 400, such as a portion of an outer surface of the flexible tubing of the fluid path set 170. The cam assembly 320 may include an inner race 322 and an outer race 324 arranged concentrically with one another. The outer race 324 may be freely rotatable relative to the inner race 322. In some aspects or examples, a plurality of rolling elements 323, such as a plurality of bearings or rollers may be provided between the inner race 322 and the outer race 324 to reduce rotating friction between the inner race 322 and the outer race 324. In the embodiment shown in FIGS. 4 and 5, the rolling elements 323 may include a plurality of rollers, such as balls or cylinders. In other aspects or examples, the rolling elements 323 between the inner race 322 and the outer race 324 may be a plain bearing, a liquid bearing, an air bearing, or the like. In other aspects or examples, the inner race 322 may bear directly against the outer race 324, for example by a low friction surface to surface contact between an outer surface of the inner race 322 and an inner surface of the outer race 324.

The inner race 322 may have a central axis L_{C} and a rotation axis L_{R}. The rotation axis L_{R} is parallel to but spaced apart from the central axis L_{C}. Thus, the inner race 322 rotates eccentrically about the rotation axis L_{R} as a shaft 332 is rotated by an associated motor 330. The rotation axis L_{R} may be fixed relative to the anvil 310, such that rotation of the inner race 322 about the rotation axis L_{R} changes the distance between the outer surface of the cam assembly 320 and the anvil 300. As such, with the flexible tubing 400 positioned on the receiving surface 312 of the anvil 310, rotation of the inner race 322 about the rotation axis L_{R} may pinch and reversibly compress the flexible tube 400 between the outer race 324 and the receiving surface 312. More particularly, rotation of the inner race 322 causes the outer race 324 to move in a direction relative to the receiving surface 312 of the anvil 310 to compress or decompress the flexible tube 400, thereby reducing or shutting off the flow path through the flexible tubing 400.

Because the outer race 324 freely rotates about the inner race 322 as the shaft 332 is rotated, the outer race 324 engages in rolling contact with the flexible tubing 400 with only minimal sliding contact between the outer surface of the outer race 324 and the flexible tubing 400. As shown in FIG. 4, as the inner race 322 is rotated to pinch the flexible tubing 400, the outer race 324 engages the flexible tube 400 at a contact point P_{C}. As the inner race 322 is further rotated towards the fully closed position shown in FIG. 5, the outer race 324 rolls along the flexible tubing 400 such that the contact point P_{C} at which the outer race 324 engages the flexible tubing 400 remains substantially constant as the flexible tubing 400 is reversibly compressed. This rolling contact between the outer race 324 and the flexible tubing 400 generates minimal frictional drag against the flexible tubing 400, thereby preventing damage to the flexible tubing 400 and extending the useable life of the flexible tubing 400. According to certain embodiments, the cammed rolling clamp of the present disclosure may apply a compression force on the flexible tube 400 between the outer race 324 and the anvil 310 that is from about 0.1% to about 100% of a rolling compression force and from about 99.9% to about 0% of a sliding compression force. In other embodiments, the rolling compression force may be from 51% to 100% and the sliding compression force may be from 0% to 49% of the force applied to the tubing 400. In still other embodiments, the rolling compression force may be from 75% to 100% and the sliding compression force may be from 0% to 25% of the force applied to the tubing 400, in further embodiments, the rolling compression force may be from 85% to 100% and the sliding compression force may be from 0% to 15% of the force applied to the tubing 400, or even form 90% to 100% rolling compression force and from 0% to 10% sliding compression force.

As used herein, the term “open”, when used in connection with the flexible tubing 400, means that an inner sidewall 402 of the flexible tubing 400 is substantially uncompressed, such that a cross sectional area of a lumen 404 of the flexible tubing 400 is the same as in a natural, relaxed state. The term “fully closed” means that the flexible tubing 400 is reversibly compressed such that diametrically opposed points P₁, P₂ of the inner sidewall 402 are brought into contact with one another, thereby reducing the cross-sectional area of the lumen 404 to substantially zero. Fluid flow is thus prohibited through the lumen 404. The term “closed” may be used interchangeably with the term “fully closed”. The terms “partially open” and “partially closed” mean that the flexible tubing 400 is reversibly compressed such that diametrically opposed points P₁, P₂ of the inner sidewall 402 are brought towards one another, reducing the cross-sectional area of the lumen 404 relative to the natural, relaxed state of the flexible tubing 400. However, the cross-sectional area of the lumen 404 when “partially open” and/or “partially closed” is greater than zero.

With continued reference to FIGS. 3 through 5, the cam assembly 320 may be connected to the motor 330 via the shaft 332. The shaft 332 may be coaxial with the rotation axis L_{R} of the inner race 322 to impart eccentric rotation to the inner race 322. The motor 330 and the anvil 310 may be attached to a mounting plate 340, which may be attachable to the housing 11 or another component of the fluid injector system 1000. The motor 330 may be a servo motor, stepper motor, or the like to allow precise control of the rotational position of the shaft 332 and the inner race 322. Rotating the inner race 322 directly from rotation of the motor 330 may allow for rapid response of the tubing clamp 300. For example, in certain procedures such as angiography injection procedures pressures as high as 1200 psi may be utilized. At injection pressures of 1200 psi and fluid flow rates of at 25 to 40 mL/sec, an air bubble can travel up to 4 feet in a tube path depending on the inner diameter (ID) of the tubing. For example, at approximately 1200 psi, an air bubble may travel a distance corresponding to 3.2 mL over 80 milliseconds at a flow rate of 30 mL/sec in a tubing with a 0.072 inch ID. The distance equivalence of 3.2 mL volume for such an embodiment may be approximately 4 ft of tubing length travelled during 80 milliseconds. In various embodiments, the tubing clamp 300 may be rotated from the open position of FIG. 4 to the fully closed position of FIG. 5 in less than about 200 milliseconds, for example from about 40 milliseconds to about 100 milliseconds or in other embodiments from about 40 milliseconds to about 80 milliseconds. This rapid response time may ensure the tubing clamp 300 can attain the closed position (FIG. 5) upon receiving a signal from the controller 200 (see FIG. 2) prior to air reaching the patient, particularly for high pressure injection procedures. In other embodiments, one or more intermediate gears may be present intermediate between the shaft 332 and the cam assembly 320, wherein the gearing ratio changes the relative rotation of the cam assembly 320 in relation to the shaft 332. In other embodiments, increasing the offset between the offset between the central axis L_{C} and rotation axis L_{R} of the inner race 322 may result in more rapid response time for the tubing clamp 300 attaining the closed position upon air bubble detection.

With continued reference to the embodiment illustrated in FIGS. 4 and 5, the central axis L_{C} and rotation axis L_{R} of the inner race 322 may extend substantially perpendicular to a longitudinal axis LT of the flexible tubing 400. In this arrangement, rotating the cam assembly 320 to the closed position may create a directional pulse of fluid within the flexible tubing 400. More particularly, the rolling contact between the outer race 324 and the flexible tubing 400 may displace the fluid in the portion of the flexible tubing 400 compressed by the outer race 324. As illustrated in FIG. 4, rotation of the inner race 322 in the direction of arrow A may cause a pulse of fluid in the direction of arrow C as the flexible tubing 400 is compressed. Conversely, rotation of the inner race 322 in the direction of arrow B may cause a pulse of fluid in the direction of arrow D as the flexible tubing 400 is compressed. As such, the tubing clamp 300 may be arranged and operated to provide the fluid pulse in a clinically desirable direction with respect to the patient and the other components of the fluid injector system 1000. For example, when closing the tubing in response to detected air, it may be desired that any potential fluid pulse may be directed upstream or away from the patient.

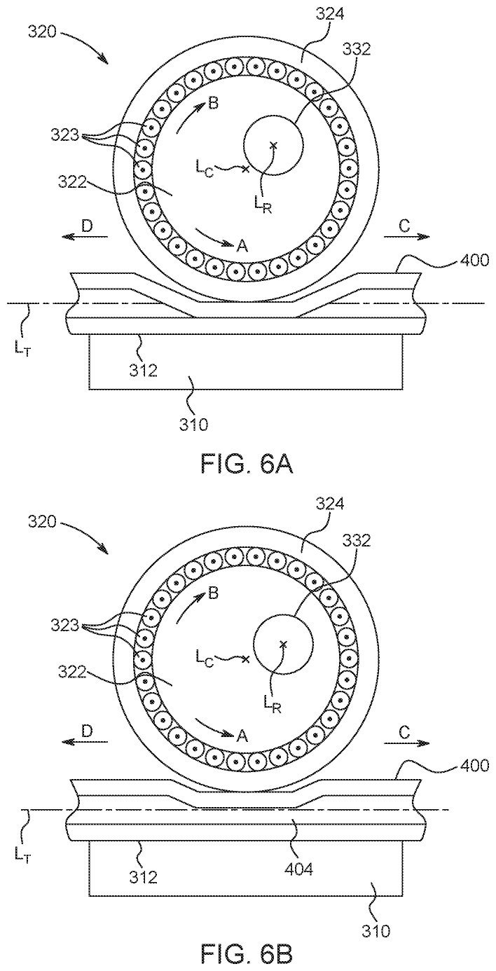

In the aspects or examples shown in FIGS. 4 and 5, the inner race may 322 be continuously rotatable, i.e. rotatable 360°, about the rotation axis L_{R} with the flexible tubing 400 positioned on the receiving surface 312 of the anvil 310. In the closed position as shown in FIG. 5, the central axis L_{C} of the inner race 322 is between the anvil 310 and the rotation axis L_{R} of the inner race 322. As such, the cam assembly 320 is in a most eccentric position when the flexible tubing 400 is fully closed. To subsequently move the cam assembly to the open position of FIG. 4, the inner race 322 may be rotated in the direction of either arrow A or arrow B about the rotation axis L_{R}. In other aspects or examples, as shown in FIGS. 6A and 6B, the inner race 322 may not rotate a full 360°. Rather, the closed position of the tubing clamp 300 is not the most eccentric position of the inner race 322. That is, with the flexible tubing 400 fully closed, the central axis L_{C} of the inner race 322 is not directly between the anvil 310 and the rotation axis L_{R} of the inner race 322. Thus, to move the cam assembly 320 from the closed position shown in FIG. 6A back to the open position, the inner race 322 must be rotated in the direction of arrow B while rotation in the direction of arrow A will further compress and potentially deform flexible tubing 400. FIG. 6B shows the cam assembly 320 and the flexible tubing 400 in a partially closed position, in which the cross-section of the lumen 404 is reduced relative to the open position. The flow path of the cross-sectional pathway may be controlled by rolling of the cam assembly 320 such that any cross-sectional flow path may be achieved, for example, ranging from 100% open to 0% open and all potential amounts in between. In other embodiment, specific cross-sectional pathway flow path values may be selected by the controller and the cam assembly 320 rolled an appropriate amount to achieve the selected flow path value. In other embodiments, the value associated with the particular rolled position of the cam assembly 320 may change over time, for example due to changes in tubing diameter or tube wall thickness. The controller may adjust and correct to these changes in real-time by utilizing flow values read by a down-stream flow rate detector to adjust the roll position of the cam assembly 320 to provide the desired cross-sectional flow path value.

In some aspects or examples, the anvil and hammer or cam may completely sever the flexible tubing 400, for example when air is detected in the tubing to prevent the air from reaching the vasculature of the patient. According to these embodiments, when the flexible tubing is severed, the pressure gradient in the distal end of the tubing that is connected to the patient is reversed, such that pressure at the catheter tip that is inside the vascular system of the patient is at some pressure greater than atmospheric pressure and the severed end of the distal portion of the tubing is at atmospheric pressure which is zero gage pressure. Fluid instantly flows from the catheter to the severed end of the tubing so air, contrast, and/or saline are all prevented from entering the patient. Upon severing of the flexible tubing, the diagnostic procedure is terminated and the sterile disposable tubing and catheter must be replaced if the diagnostic procedure is to continue. According to these embodiments, severing the tubing may be considered a fail-safe protocol to prevent detected air from reaching the patient.

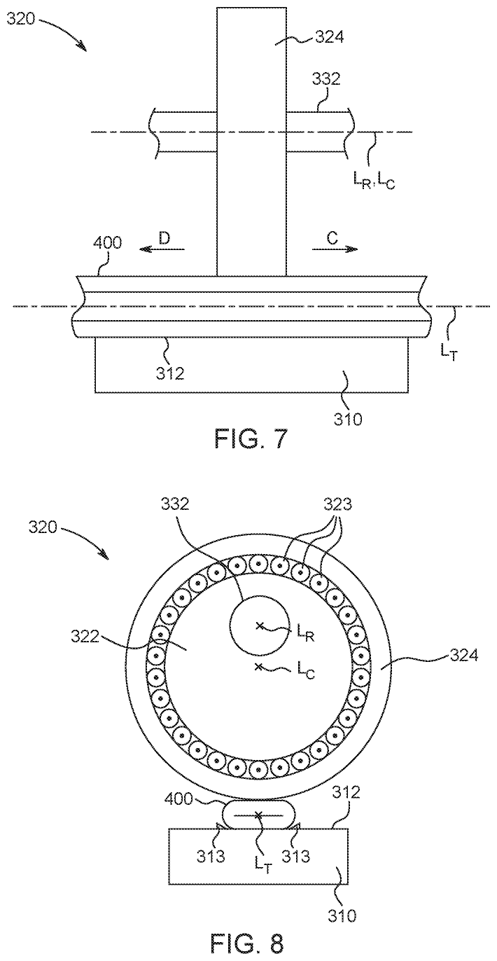

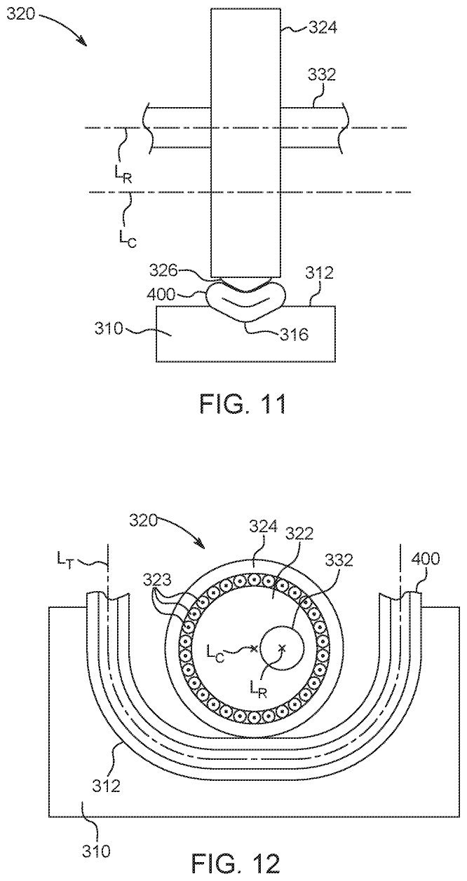

Referring now to FIGS. 7 and 8, in other aspects or examples of the tubing clamp 300, the central axis L_{C} and the rotation axis L_{R} of the inner race 322 extend substantially parallel to the longitudinal axis LT of the flexible tubing 400. As the rolling contact occurs transverse to the longitudinal axis LT of the flexible tubing 400, the fluid pulse may be substantially balanced or equalized between the direction of arrow C and the direction of arrow D regardless of the direction in which the inner race 322 is rotated. The arrangement of FIGS. 7 and 8 may thus be used when a substantially balanced or equalized pulse is clinically desirable. In addition, the example of FIGS. 7 and 8 may induce a smaller pulse than the example of FIGS. 4 and 5 (assuming an otherwise dimensionally identical cam assembly 320) as the example of FIGS. 7 and 8 may compress a relatively smaller section of the flexible tube 400. The anvil 310 may include one or more guide tabs 313 projecting up from the receiving surface 312 to keep the flexible tubing 400 in position on the anvil 310 as the cam assembly 320 rolls transversely across the flexible tubing 400 between the open and closed positions. The one or more guide tabs 313 may be arranged to retain the flexible tubing 400 in position against a force perpendicular to the longitudinal axis LT of the flexible tubing 400. However, the one or more guide tabs 313 should not inhibit reversible compression of the flexible tubing 400 between the outer race 324 and the anvil 310. In some aspects or examples, the anvil 310 may define a groove, as described herein in connection with FIGS. 10 and 11, to keep the flexible tubing 400 in position on the anvil 310 as the cam assembly 320 rolls transversely across the flexible tubing 400 between the open and closed positions. In some aspects or examples, the flexible tubing 400 may be secured to the anvil 310 with clips or other fasteners. In still other embodiments, one or more surface textures on receiving surface 312 may prevent undesired movement of the flexible tubing 400 during transverse movement of the cam assembly 320. While embodiments of the clamp having the central axis L_{C} and the rotation axis L_{R} of the inner race 322 extending substantially parallel to or perpendicular to the longitudinal axis L_{T} of the flexible tubing 400 are illustrated, other angles between the central axis L_{C} and the rotation axis L_{R} of the inner race 322 and the longitudinal axis LT of the flexible tubing 400 are contemplated and are within the scope of the present disclosure.

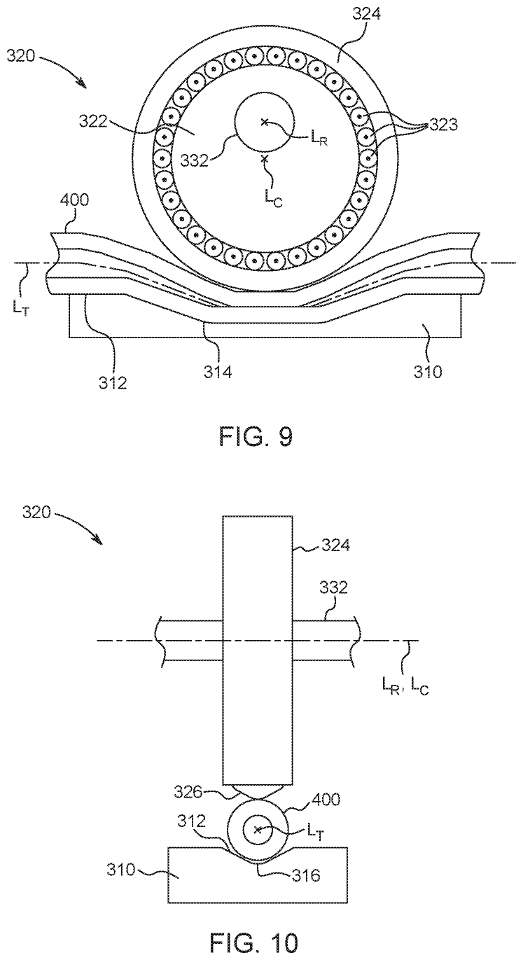

Referring now to FIG. 9, another aspect or example of the tubing clamp 300 is shown in which the receiving surface 312 of the anvil 310 defines at least one detent 314 for receiving the outer race 324 in the closed position. According to this embodiment, the outer race 324 may extend at least partially into the detent 314 when the tubing clamp 300 is in the closed position. The detent 314 may physically inhibit reverse rotation of the cam assembly 320. As such, inadvertent opening of the tubing clamp 300 may be prevented, for example due pressure within the flexible tubing 400 or to power loss to the motor 330. In some aspects or examples, the detent 314 may retain the outer race 324 and the flexible tubing 400 in the closed position even in the absence of any current being supplied to the motor 330. Thus, in certain embodiments the fit between the outer race 324 and the detent 314 may be sufficient to prevent fluid pressure within the flexible tubing 400 from forcing the cam assembly 320 to the open position. The geometry of the detent 314 may be selected such that a predetermined rotational force, corresponding to a predetermined current supplied to the motor 330, is required to disengage the outer race 324 from the detent 314 to return the tubing clamp 300 to the open position. Other than the detent 314, the tubing clamp 300 of FIG. 9 may be essentially similar to the tubing clamp 300 of FIGS. 4 through 8.

Referring now to FIGS. 10 and 11, another aspect or example of the tubing clamp 300 is shown in which the receiving surface 312 of the anvil 310 defines a groove or channel 316 for indexing and/or retaining the flexible tubing 400 in a particular position relative to the anvil 310 and the outer race 324 of the cam assembly 320. In certain embodiments, the groove or channel 316 may be arcuate and have a radius substantially equal to or greater than an outer diameter of the flexible tubing 400. According to various embodiments, the outer race 324 includes an annular protrusion 326 that is complementary to and cooperates with the groove or channel 316 to compress the flexible tube 400 into the groove or channel 316 when the tubing clamp 300 is in the closed position. Apart from the groove or channel 316 and the annular protrusion 326, the tubing clamp 300 of FIGS. 10 and 11 may be essentially similar to the tubing clamp 300 of FIGS. 4 through 8.

Referring now to FIG. 12, another aspect or example of the tubing clamp 300 is shown in which the receiving surface 312 of the anvil 310 is curved parallel to the longitudinal axis L_{T} of the flexible tube 400 such that the flexible tube 400 at least partially surrounds or wraps around the cam assembly 320. The curved receiving surface 310 provides additional engagement area with the flexible tubing 400 and provides visual and/or tactile feedback to the medical practitioner that the flexible tube is properly positioned in the tubing clamp 300.

In any of the various aspects or examples shown in FIGS. 4 through 12, the outer race 324 may include surface features on the outer surface to improve engagement with the flexible tubing 400. For example, the surface of the outer race 324 may have a textured surface to increase the grip of the outer race 324 against the flexible tubing 400. According to certain embodiments, the textured surface outer race 324 may have a surface roughness in a range of between 2 microinches and 125 microinches, or in other embodiments, in a range between 20 microinches and 75 microinches. In some aspects or examples, the textured surface of the outer race 324 may be directional such that the outer race 324 may be more difficult to roll off of the flexible tubing 400 than to roll onto the flexible tubing 400. As such, the textured surface of the outer race 324 may induce the outer race 324 to remain in the closed position. According to other embodiments, the receiving surface 312 of the anvil 310 may include a textured surface configured to assist in maintaining the position of the flexible tubing 400 on the receiving surface 312 of the anvil 310.

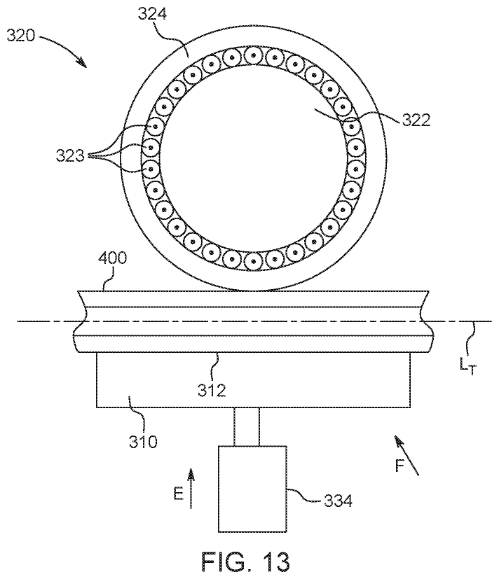

In the aspects or examples discussed in connection with FIGS. 4 through 12, the inner race 322 is rotated to move the outer race 324 towards the anvil 310. In other aspects or examples, as shown in FIGS. 13 through 15, the anvil 310 may be moved toward the cam assembly 320, optionally with the inner race 322 remaining stationary. For example, the outer race 324 may rotate relative to the inner race 322 to provide rolling contact at the contact point P_{C} against the flexible tubing 400, in substantially the same manner described in connection with FIGS. 4 through 8. In the example shown in FIG. 13, the anvil 310 may be moved perpendicular to the receiving surface 312 in the direction of arrow E to at least partially compress the flexible tubing 400 between the anvil 310 and the outer race 324, for example when moving the tubing clamp 300 to the closed position. In other aspects or examples, the anvil 310 may be moved at an angle with respect to the receiving surface 312 in the direction of arrow F to compress the flexible tubing 400 between the anvil 310 and the outer race 324. In still other embodiments, the anvil 310 may be associated with a compressible member 334, such as a spring, that forces the anvil 310 towards the cam assembly 320 by a force in the direction E associated with the spring force constant of the system. According to these embodiments, the spring force constant of compressible member 334 may be selected to ensure closure of the flexible tubing 400 when the system is in the closed position while ensuring that the compressive force applied does not deform or permanently alter the flexible tubing 400.

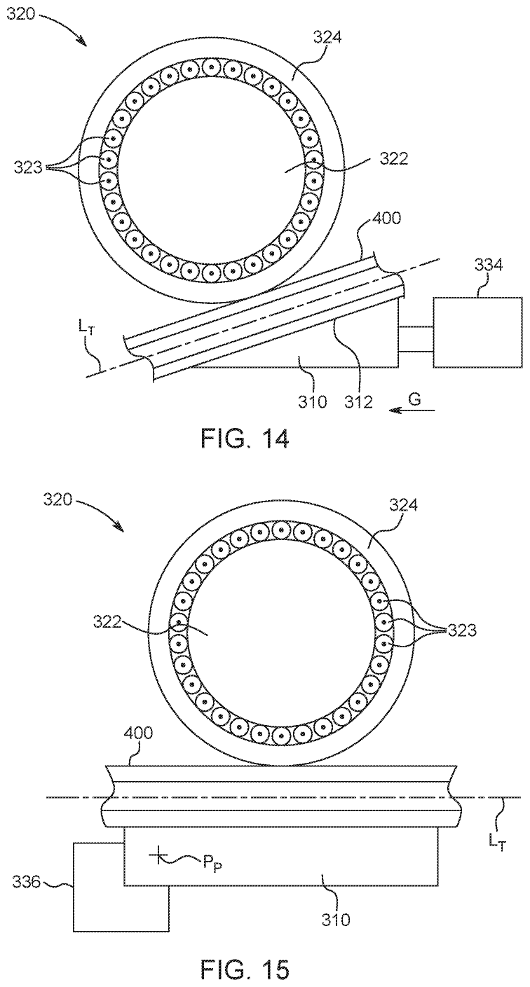

In the example shown in FIG. 14, the anvil 310 may be a wedge or ramp that is moved in the direction of arrow G to further compress the flexible tubing 400 between the anvil 310 and the outer race 324. In the examples shown in FIGS. 13 through 14, the anvil 310 may be moved by a linear motor or spring 334, such as a linear actuator, a lead screw, or the like. In the example shown in FIG. 15, the anvil 310 may be rotated about a pivot point P_{P} to compress the flexible tubing 400 between the anvil 310 and the outer race 324. The anvil 310 may be connected to a motor 336, such as a stepper motor, servo motor, or the like, at the pivot point Pp.

The aspects and examples of the tubing clamp 300 described in connection with FIGS. 3 through 16 may be configured and used as a shut-off valve, a flow rate control valve, or a combination thereof. When configured as a shut-off valve, the inner race 322 of the tubing clamp 300 may be rotated between the open position, in which the flexible tubing 400 is substantially uncompressed, and the closed position, in which the flexible tubing 400 is fully compressed. The term “fully compressed”, as used herein in reference to tubing, means that at least a region of the flexible tubing 400 is compressed such that an internal lumen of the tubing is reduced to a cross sectional area of zero or a negligible value through which fluid cannot flow.

When configured as a flow rate control valve, the tubing clamp 300 may be rotated between a plurality of partially open and/or partially closed positions to reversibly compress the flexible tubing 400 by varying degrees. For example, the inner race 322 may be rotated to any of a finite or infinite number of positions between the open position and the fully closed position and thus, provide control of the fluid flow through the reduced lumen cross-section. The cross-sectional area of the internal lumen of the flexible tubing 400 may therefore be controlled depending on the rotational position of the inner race 322. According to certain embodiments, the cross-sectional area of the lumen 404 of the flexible tubing 400 may be chosen to correspond to a known and/or empirically-derived flow rate of fluid through the flexible tubing 400. In some aspects or examples, the tubing clamp 300 may include an encoder to determine the position of the inner race 322, and the controller 200 may be configured to actuate the motor 330 to rotate the inner race 322 to a desired position associated with a desired flow rate of fluid through the flexible tubing 400. In some aspects or examples, the fluid injector system 1000 may include at least one flow rate sensor downstream of the tubing clamp 300 to measure the flow of fluid through the tubing clamp 300. The controller 200 may actuate the motor 330 to rotate the inner race 322 to achieve a desired flow rate as measured by the at least one flow rate sensor.

In some aspects or examples, the tubing clamp 300 may be controlled by the controller 200 based on motor current. In particular, the controller 200 may be configured to rotate the inner race 324 until a predetermined current is drawn by the motor 330, corresponding to the closed position, or any other at least partially compressed position, of the cam assembly 320. Measurement of the motor current may take into account creep experienced over the useable life of the flexible tubing 400, which may change the force required to compress the flexible tubing 400. For example in certain embodiments, the controller may utilize the flow values measured at the at least one downstream flow rate sensor to benchmark the motor current and position of the cam assembly 320 with the flow rate of fluid in the downstream fluid path and update amount of rotation necessary to achieve a desired flow rate or to fully compress the flexible tubing 400.

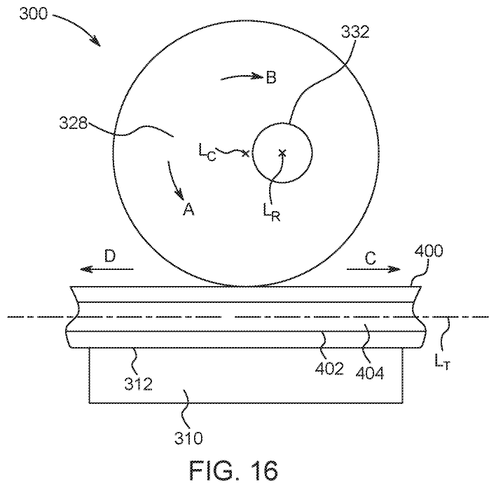

In the aspects and examples of the tubing clamp 300 described in connection with FIGS. 3 through 15, the cam assembly 320 includes an inner race 322 and an outer race 324 relative to the inner race 322. Referring now to FIG. 16, in some aspects or examples, the tubing clamp 300 may include a rollable solid cam 328 mounted eccentrically to the shaft 332, in place of the cam assembly 320 of the aspects of FIGS. 3 through 15. Rotation of the shaft 332 about the rotation axis L_{R} may change the distance between the outer surface of the rollable solid cam 328 and the receiving surface 312 of anvil 300. As such, when the flexible tubing 400 is positioned on the receiving surface 312 of the anvil 310, rotation of the shaft 332 about the rotation axis L_{R} may cause the rollable solid cam 328 to move in a direction relative to the receiving surface 312 of the anvil 310 to compress or decompress the flexible tube 400, thereby reducing or shutting off the flow path through the lumen 404. Other than the rollable solid cam 328, the features and operation of these examples may be essentially identical to the examples of FIGS. 3 through 15.

According to certain embodiments, the tubing clamp 300 of FIG. 16 may apply a compression force on the flexible tube 400 between the rollable solid cam 328 and the anvil 310 that is from about 0.1% to about 100% of a rolling compression force and from about 99.9% to about 0% of a sliding compression force. In other embodiments, the rolling compression force may be from 51% to 100% and the sliding compression force may be from 0% to 49% of the force applied to the tubing 400.

While examples of fluid injector systems, tubing clamps, and methods of operation thereof were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A clamp for regulating fluid flow in a flexible tube of a fluid injector system, the clamp comprising:
an anvil comprising a receiving surface for receiving at least a portion of the flexible tube; and
a cam assembly comprising:
a rollable outer race; and
an inner race having a central axis and a rotation axis spaced apart from
the central axis, wherein the inner race is rotatable relative to the outer
race about the central axis,
wherein rotation of the inner race about the rotation axis rolls the outer race in a direction relative to the receiving surface of the anvil to reversibly compress the flexible tube between the outer race and the anvil.

2. The clamp of claim 1, wherein the inner race of the cam assembly is reversibly rotatable to:
a first position in which the flexible tube is substantially uncompressed between the outer race and the anvil such that a fluid can flow through a lumen of the flexible tube; and
a second position in which the lumen of the flexible tube is fully compressed between the outer race and the anvil to prohibit fluid flow through the flexible tube, such that fluid communication across a compressed region of the flexible tube is blocked.

3. The clamp of claim 1, wherein the inner race of the cam assembly is reversibly rotatable to at least partially compress the flexible tube between the outer race and the anvil, thereby controlling a flow rate of fluid through an at least partially compressed region of the flexible tube.

4. The clamp of claim 1, wherein a compression force on the flexible tube between the outer race and the anvil is from about 0.1% to about 100% of a rolling compression force and from about 99.9% to about 0% of a sliding compression force.

5. The clamp of claim 1, wherein the cam assembly further comprises a plurality of rolling elements between the inner race and the outer race.

6. The clamp of claim 1, wherein the central axis and the rotation axis of the inner race extend perpendicular to a longitudinal axis of the flexible tube.

7. The clamp of claim 1, wherein the central axis and the rotation axis of the inner race extend parallel to a longitudinal axis of the flexible tube.

8. The clamp of claim 1, wherein the receiving surface of the anvil defines a groove for receiving at least a portion of the flexible tube.

9. The clamp of claim 8, wherein the outer race comprises at least one annular protrusion cooperative with the groove of the receiving surface.

10. The clamp of claim 1, further comprising a motor having a shaft for rotating the inner race about the rotation axis.

11. The clamp of claim 1, wherein at least a portion of the outer race has a textured surface to prevent slippage between the outer race and flexible tube.

12. The clamp of claim 11, wherein the textured surface of the outer race is directional to prevent rotation of the outer race away from the flexible tube.

13. The clamp of claim 1, wherein the anvil defines a detent, and
wherein rotation of the inner race about the rotation axis compresses the flexible tube between the outer race and the detent of the anvil.

14. The clamp of claim 1, wherein at least a portion of a section of the receiving surface of the anvil is substantially planar.

15. The clamp of claim 1, wherein a contact point of the outer race at which the outer race engages the flexible tube remains in constant contact with the flexible tube as the flexible tube is reversibly compressed.

16. A fluid injector system comprising:
at least one injector for pressurizing and delivering at least one fluid from at least one fluid reservoir;
a fluid path set comprising at least one flexible tube, wherein the fluid path set is in fluid communication with the at least one fluid reservoir;
a controller; and
at least one clamp for regulating fluid flow through the at least one flexible tube of the fluid path set, the at least one clamp comprising:
an anvil comprising a receiving surface for receiving at least a portion of the at least one flexible tube; and
a cam assembly comprising:
a rollable outer race; and
an inner race having a central axis and a rotation axis spaced apart from the central axis, wherein the inner race is rotatable relative to the outer race about the central axis,
wherein rotation of the inner race about the rotation axis rolls the outer race in a direction relative to the receiving surface of the anvil to reversibly compress the flexible tube between the outer race and the anvil, and
wherein the controller is programmed or configured to control rotation of the inner race of the cam assembly.

17. The fluid injector system of claim 16, wherein the controller is programmed or configured to reversibly rotate the inner race of the cam assembly to:
a first position in which the flexible tube is substantially uncompressed between the outer race and the anvil such that fluid can flow through a lumen of the flexible tube; and
a second position in which the lumen of the flexible tube is fully compressed between the outer race and the anvil to prohibit fluid flow through the flexible tube, such that fluid communication across a compressed region of the flexible tube is blocked.

18. The fluid injector system of claim 16, wherein the controller is programmed or configured to reversibly rotate the inner race of the cam assembly to at least partially compress the flexible tube between the outer race and the anvil, thereby controlling a flow rate of the at least one fluid through an at least partially compressed region of the flexible tube.

19. The fluid injector system of claim 17, further comprising:
at least one air detector for detecting a presence of air in the fluid path set,
wherein the at least one clamp is located downstream of the at least one air detector, and
wherein the controller is programmed or configured to rotate the inner race of the cam assembly to the second position in response to detecting the presence of air in the fluid path set by the at least one air detector to prevent flow of the air past the at least one clamp.

20. The fluid injector system of claim 17, wherein the controller is further programmed or configured to rotate the inner race of the cam assembly to the second position to prevent backflow of at least one first fluid into one or more of at least one second fluid reservoir or a second fluid tube.

21-24. (canceled)
